(19) United States
(12) Patent Application Publication
(10) Pub. No.: US 2019/0015174 A1
(43) Pub. Date: Jan. 17, 2019

(54) FLUOROSCOPIC SURGICAL MARKING SYSTEM

(71) Applicant: Roland Jones, Tallahasee, FL (US)

(72) Inventor: Roland Jones, Tallahasee, FL (US)

(21) Appl. No.: 15/646,495

(22) Filed: Jul. 11, 2017

(51) Int. Cl. A61B 90/00

(52) U.S. Cl. CPC A61B 90/39; A61B 2090/3966; A61B 2090/376; A61B 2090/3983; A61B 2090/3908; A61B 2090/3933

(57) ABSTRACT

The embodiments provide a surgical marking system for measuring and marking the location of various anatomical landmarks/bones on patient's skin before a surgical procedure. The system includes a shaft having a plurality of metallic markings and a radiographical pointer tip which is visible on fluoroscopic x-ray images. The shaft further includes a fluid reservoir for marking the location of an incision so that it is visible through fluoroscopic imaging technology and which provides a temporary marking agent that readily disappears after the surgical procedure has completed.

### FIELD

The present invention relates to a surgical marking system, and in particular, to a surgical marking system including a rectangular-shaped housing having a arrow-head point attached to x-ray opaque metallic ruler and further containing a fluoroscopically visible marking agent to mark, measure, and identify anatomical landmarks on a patient's skin in preparation of a surgical procedure.

### BACKGROUND

Surgical marking pens have been used for decades in the United States. The pens are primarily used by a surgeon or surgical technician to mark the incision location on the patient's body to determine where and how long the cut is to be made during a surgical procedure. These marking lines are very important in specialties such as orthopedic or plastic surgery where uniform and linear marking are essential to surgical success.

Most commonly, a surgeon or nurse will use a felt tip pen to place a circle or small lines at the incision location indicating where and how long the cut is to be made. However, if the pen is applied too firmly or the skin of the patient (i.e., tissue surface) is moist, the marker will likely “bleed” after being applied. Further, during the surgical procedure blood may run onto the mark which is then wiped, possibly eliminating any indication of where the mark was originally applied.

In addition, many of these marking pens are used only once and thrown out because of the risk of contaminates or because they are left uncovered and dry out. In many cases, the surgical staff is forced to improvise by wetting the marker tip during surgery which often leads to contamination or blurring pre-selected surgical marking when re-applied to the patient's skin.

Though there are several references which disclose a surgical marking pen such as U.S. PAT. App. No 2008/0269694 to Viscot Medical, LLC; U.S. Pat. App. No. 2008/0287978 to Hickham; and U.S. Pat. App. No. 2010/0292714 to Shah; there is no single apparatus or system which includes a metallic measuring means about the exterior surface while further containing a marking agent or pointer which is visible on a fluoroscopic X-ray.

### SUMMARY OF THE INVENTION

The embodiments provided disclose a fluoroscopic surgical marking system for use in preparation of a surgical procedure to place a thin uniformly applied fluoroscopic marking agent on the patient's skin, and provide measurements of internal anatomical structures, which are visible on x-ray images. The system includes: a rectangular-shaped housing configured to provide to a stable base when placed on a patient's body between markings; a pen cap at the first end to prevent the ink reservoir from drying out and provide a sterile environment when hermetically sealed onto the first end of the housing; and an ink reservoir contained within the housing and filled with a fluoroscopic marking agent.

The system further includes an arrow-head pointer located on an exterior portion of a tapered surface at the first end of the housing to identify and emphasize specific anatomical landmark along the patient's body and provide accurate x-ray visible measurements critical during the perioperative phases. Further, located adjacent to the arrow-head marking tip (i.e., pointer) is a felt tip marker which allows the patient's skin to be marked with a fluoroscopic marking agent contained within the ink reservoir.

### BRIEF DESCRIPTION OF THE DRAWINGS

A more complete understanding of the embodiments, and the attendant advantages and features thereof, will be more readily understood by reference to the following detailed description when considered in conjunction with the accompanying drawings wherein:

FIG. 1 is a perspective view of a fluoroscopic surgical marking system;

FIG. 2 is cross-sectional view of the system;

FIG. 3 is an exploded view of the system;

FIG. 4 is a view of the arrow tip pointer and metallic ruler; and

FIG. 5 is a view of the metallic ruler and pen shaft.

### DETAILED DESCRIPTION

The specific details of the single embodiment or variety of embodiments described herein are set forth in this application. Any specific details of the embodiments are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

The embodiments provide a surgical marking and measurement system which allows surgeons and surgical staff to measure and mark a patient's skin in preparation of a surgical procedure. The system 10 includes an arrow-head pointer affixed on the exterior surface of a taper portion of the first end of the housing and continuing along the planar rectangular-shaped housing with pre-determined galvanized incremental metallic marking throughout the length of the housing. Further contained with the hollow housing is an ink reservoir which contains a fluoroscopic marking agent which enables a surgeon to uniform apply the marking agent along the patient's skin without the blurring found with traditional surgical marking devices.

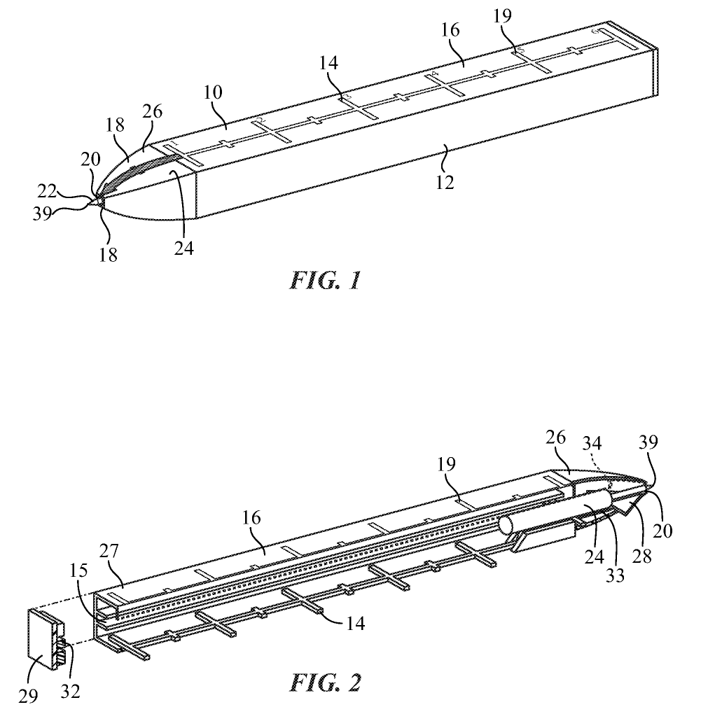

Referring now to the drawings wherein like reference numerals designate identical or corresponding parts throughout the views. There is shown in FIG. 1 a fluoroscopic surgical marking system 10 including: a rectangular-shaped housing 12; a galvanized interior metallic ruler 14 within an interior hollow portion of the housing 15; a tapered first end 18; and ink reservoir 24.

The rectangular-shaped housing 12 is specifically formed with the intent to enable the surgeon to rest the system 10 on patient body between uses during a surgical procedure with rolling on the floor and accumulating contaminates. The rectangular-shaped housing 16 includes a first exterior surface 16 having an exterior ruler 19 extending the length of the housing which allows surgeons to measure exterior landmarks along the patients skin which are not intended to be visible on an x-ray image. Further located at the first end of the rectangular-shaped housing 26 is the tapered first end 18 which is downwardly angled from the planar surface of the rectangular-shaped housing 27 to a dimensioned aperture 20 proportioned to fit the apex of a felt tip pen 39 to protrude outwardly from the ink reservoir 24 and selectively mark the patient's body with the fluoroscopic marking agent 22.

It is contemplated the rectangular-shaped housing 12 is approximately 6-8 inches in length, one inch wide, and has a ½ inch height and ergonomically dimensioned be held in a surgeon's hand in a similar manner as a traditional writing instrument. It is contemplated the system 10 material such as a plastic or thermoplastic which is designed to delivered in a sterile packaging to the operating room in anticipation of a medical procedure.

Shown in FIG. 2 is a cross-sectional view of the system 10 showing a hollow grooved interior hollow portion 15 of the rectangular-shaped housing 12 and further including a metallic ruler 14 comprised of a galvanized metal such as zinc, iron, or carbon with a plurality of pre-determined incremental measurements extending from the first end of the rectangular-shaped housing 26 to the second end 27 and super imposed directly below the exterior ruler 19.

The metallic ruler 14 is further includes arrow-head pointer tip 28 at the tapered first end 18 which is visible in x-ray images and used to emphasize and distinguish a specific landmark. Contrary to current paper measuring devices susceptible to tearing or binding when wet, the metallic ruler 14 is contained within the hollow interior portion 15 and which is hermetically sealed from any water, perspiration, or blood which may obstruct an incremental reading.

Further illustrated in FIG. 2 is the releasably attachable end cap 29 positioned at the second end of the housing 27 which allows a user to access the hollow interior portion 15 and ink reservoir 24. If a re-fill or replacement of the ink reservoir 24 is necessary, the user may simply pull the end cap 29 away from the second end 27 which are releasably attached at male-female fitting 32.

The ink reservoir 24 is located within a grooved portion of the metallic ruler 33 at the rear of the arrow-head pointer tip 28 and nuzzled between the grooved portion of the metallic ruler 33 and a vertical partition 34 which extends the length of the rectangular-shaped housing 12.

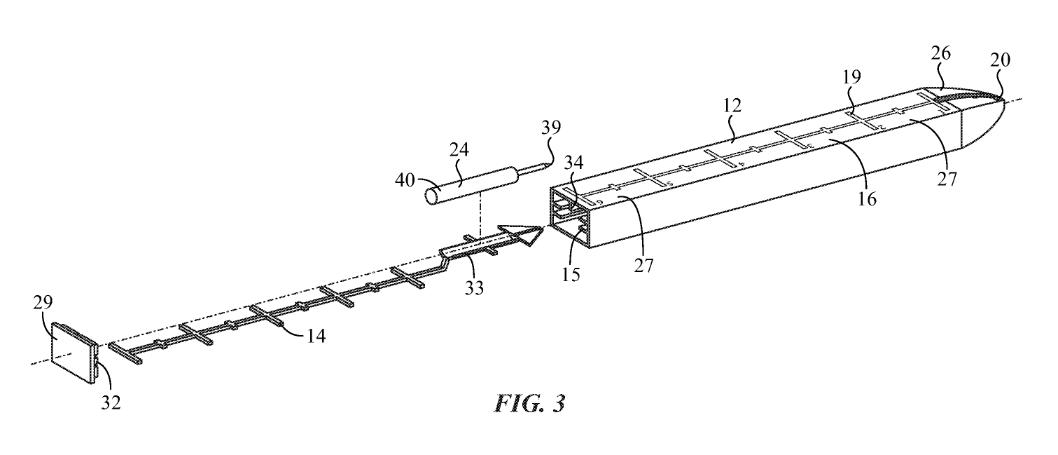

Shown in FIG. 3 is an exploded view of the system 10 including the hollow grooved interior portion 25 of the housing which acts to guide and seat the ink reservoir 24 during manufacture and replacement. The grooves further act to ensure the felt tip marker 39 and ink reservoir remains steady during use and marking agent 40 is uniformly applied to the patient's skin at the felt tip marker 39. The system 10 is designed to allow the fluoroscopic marking agent to selectively apply a uniform layer of ink at the designated location in preparation for incisions or small perforations. The felt tip marker 39, when depressed on the patient's skin, allows fluoroscopic ink to evenly flow out the tip to prevent smudging or further non-linear distortions during surgery, or creating a blurry non-linear line within an x-ray image.

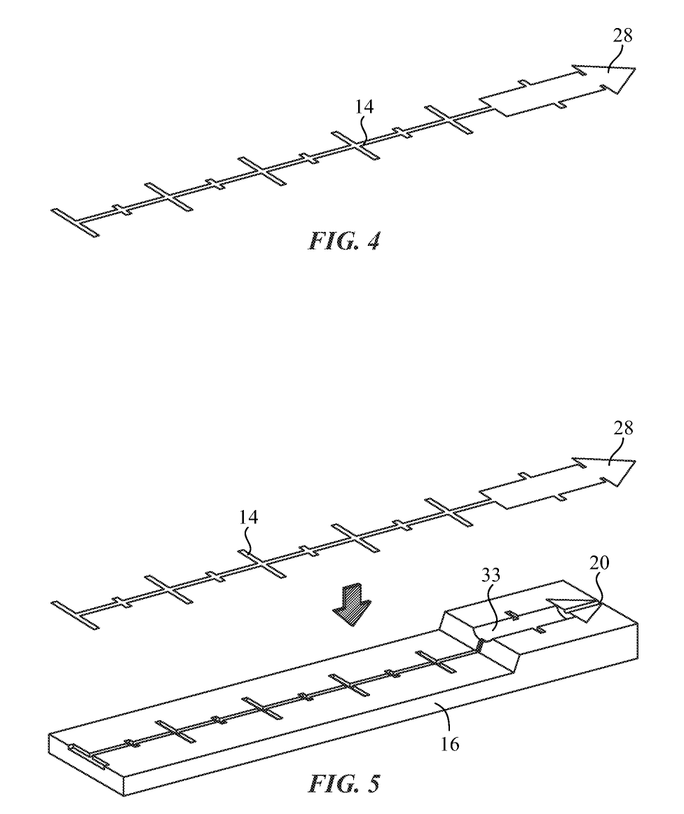

Shown in FIG. 4 and FIG. 5 is a detailed and exploded view of the galvanized metallic ruler 14 and arrow tip pointer 28 (i.e., projectile point) at the first end of the housing 26 and which has an approximate length of one and a half inches and width of ¼ inches.

It will be appreciated by persons skilled in the art that the present embodiment is not limited to what has been particularly shown and described hereinabove. In addition, unless mention was made above to the contrary, it should be noted that all of the accompanying drawings are not to scale. A variety of modifications and variations are possible in light of the above teachings without departing from the following claims.

What is claimed is:

1. A surgical marking system, the system comprising:
an elongated housing having a substantially planar first side including a plurality of metallic marking and further including a fluoroscopic marking agent to enable the marking agent to be visible in X-ray imaging.

2. The system of claim 1, further including a radio-opaque arrow-head marker at a taper surface at a first end of the elongated housing and configured to identify and emphasize an internal anatomic landmark visible on an x-ray image when placed a patient's skin.

3. The system of claim 2, wherein the x-ray opaque measuring means is affixed to a portion the radio-opaque arrow head mark.

4. The system of claim 1, wherein the x-ray opaque measuring means is comprised of an x-ray visible galvanized metal.

5. The system of claim 1, further including a releasably attachable ink cap at a first end to provide a hermetical seal with the elongated housing when releasably attached to the elongated housing to prevent a contained fluoroscopic marking agent from dissipating during use.

6. The system of claim 3, wherein the first end of the housing further includes an outward protruding felt tip marker configured to apply a uniform layer of the fluoroscopic marking agent on the patient's skin.

7. The system of claim 1, wherein a second end of the housing further includes a semi-cylindrically shaped end cap to enable replacement of a contained ink reservoir within a grooved internal portion of the elongated housing.

8. The system of claim 4, wherein the grooved internal portion of the elongated housing extends from a first end of the housing to a second end of housing and is configured to guide and stabilize the ink reservoir into a dimensioned tip of the housing during replacement.

9. The system of claim 1, wherein the housing is rectangular shaped to provide stability when placed on the tissue surface during use.

10. A marking system for use in preparation of a surgical procedure, the system comprising:
a substantially elongated and rectangular-shaped housing having a metallic ruler attached to an x-ray opaque arrow head pointer along a tapered portion of a first end and further including a felt tip marker to provide a fluoroscopic mark agent along a selected portion of a patient's skin in preparation of a surgical procedure.

11. The system of claim 10, wherein the first end further a felt tip marker protruding outward from an aperture of the rectangular-shaped housing and configured to apply a uniform layer of a fluoroscopic marking agent on the patient's skin.

12. The system of claim 10, wherein a system further includes an ink reservoir contained in a grooved interior portion of the rectangular-shaped housing.

13. The system of claim 10, wherein a first end of the grooved internal portion further includes a set of curved stability fins to provide an enhanced stability to the ink reservoir when seated in the grooved interion portion of the rectangular-shaped housing to allow for a uniform application of the fluoroscopic marking agent to the felt tip.

14. The system of claim 10, wherein the plurality of equilaterally space metallic mark means is comprised of an x-ray opaque metal to provide an image-guided fluoroscopic measurement to a patient's internal anatomical structures.

15. The system of claim 9, wherein a second end of the rectangular-shaped housing includes a releasably attachable semi-cylindrically shaped end cap to enable access to the ink reservoir.

16. The system of claim 9, wherein the system includes a releasably attachable end cap dimensioned to provide a hermetical and sterile seal to the felt tip marker to prevent dissipation of the marking agent.

17. A marking system for use in preparation of a surgical procedure, the system comprising:
an elongated and rectangular-shaped housing having galvanized metallic ruler along one planar surface of the rectangular-shaped housing and a first end; the first end having a taper surface and arrow-head pointer to provide a referenced point of at least an anatomical landmark on a patient's skin which is visible on an x-ray image.

18. The system of claim 17, further including a felt tip marker to provide a uniform layer of a fluoroscopic marking agent on the patient's skin.

19. The system of claim 17, further including an ink reservoir contained in a plurality of grooves in the interior portion of the rectangular-shaped housing.

20. The system of claim 17, further including a dimensioned end cap at the first end to provide a sterile and hermetic seal to the felt tip marker when releasably attached to a first end of the rectangular-shaped housing.
